(19) United States
(12) Patent Application Publication
(10) Pub. No.: US 2012/0269871 A1
(43) Pub. Date: Oct. 25, 2012

(54) SOLID STATE FORMS OF RASAGILINE SALTS

(75) Inventors: Nilesh Sudhir Patil, Maharashtra (IN); Haushabhau Shivaji Pagire, Maharashtra (IN); Neela Praveen Kumar, Andhra Pradesh (IN); Nitin Sharadchandra Pradhan, Maharashtra (IN)

(73) Assignee: ACTAVIS GROUP PTC EHF, Hafnarfjordur (IS)

(21) Appl. No.: 13/505,306

(22) PCT Filed: Dec. 29, 2010
(86) PCT No.: PCT/IB10/03468

§ 371 Date: Jul. 9, 2012

(30) Foreign Application Priority Data
Dec. 30, 2009 (IN) 3241/CHE/2009

(51) Int. Cl. A61K 31/135; A61P 25/30; A61K 9/14; C07C 211/42; A61P 25/00

(52) U.S. Cl. 424/400; 564/428; 514/567

(57) ABSTRACT

Provided herein are novel crystalline forms of rasagiline salts, processes for their preparation, pharmaceutical compositions, and method of treating thereof. The rasagiline salts include a maleate salt, a mandelate salt, or a salicylate salt.

### CROSS REFERENCE TO RELATED APPLICATION

This application claims the benefit of priority to Indian provisional application No. 3241/CHE/2009, filed on Dec. 30, 2009, which is incorporated herein by reference in its entirety.

### FIELD OF THE DISCLOSURE

The present disclosure relates to novel crystalline forms of rasagiline salts, processes for their preparation, pharmaceutical compositions, and method of treating thereof.

### BACKGROUND

U.S. Pat. No. 5,532,415 discloses R-(+)-N-propargyl-1-aminoindan (rasagiline) and its pharmaceutically acceptable salts, processes for their preparation, pharmaceutical compositions, and methods of use thereof. Rasagiline has been shown to be a selective inhibitor of the B-form of the enzyme monoamine oxidase (MAO-B), useful in treating Parkinson's disease and various other conditions by inhibition of MAO in the brain. Rasagiline has the molecular formula of C₁₂H₁₃N, a molecular weight of 171.24 and a structural formula of:

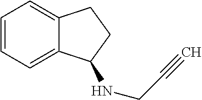

The mesylate salt of rasagiline is a selective and potent irreversible inhibitor of the B-form of the enzyme monoamine oxidase and is sold by Teva under the brand name Azilect. Rasagiline mesylate is a white to off-white powder, freely soluble in water or ethanol and sparingly soluble in isopropanol.

Various processes for the preparation of rasagiline, its enantiomers and related compounds, and their pharmaceutically acceptable salts are disclosed in U.S. Pat. Nos. 5,532,415 and 7,547,806; and U.S. Patent Application No. 2010/0234636; European Patent No. 0812190; and PCT Publication No. WO 2007/061717.

U.S. Pat. No. 5,532,415 (hereinafter referred to as the '415 patent) discloses rasagiline and pharmaceutically acceptable acid addition salts thereof. While the '415 patent mentions that pharmaceutically acceptable acid addition salts of rasagiline can be prepared by reacting the rasagiline free base with the desired acids in the presence of a suitable solvent by conventional methods, only the hydrochloride salt had been prepared and isolated.

European Patent No. 0812190 (hereinafter referred to as the '190 patent) discloses various pharmaceutically acceptable acid addition salts of rasagiline such as the mesylate, maleate, fumarate, tartrate, hydrochloride, hydrobromide, esylate, p-toluenesulfonate, benzoate, acetate, phosphate and sulfate salts, and pharmaceutical compositions comprising the rasagiline salts, and also characterizes those salts by melting points. While the '190 patent mentions that pharmaceutically acceptable acid addition salts of rasagiline can be prepared by reacting the rasagiline free base with the desired acids in the presence of a suitable solvent by conventional methods, only the mesylate and hydrochloride salts had been prepared.

U.S. Pat. No. 7,547,806 discloses the rasagiline tannate salt, a process for its preparation and pharmaceutical compositions comprising the rasagiline tannate.

U.S. Patent Application No. 2010/0234636 discloses the edisilate and oxalate salts of rasagiline, processes for their preparation, pharmaceutical compositions comprising the salts, and characterizes the salts by powder X-ray diffraction (P-XRD).

PCT Publication No. WO 2007/061717 discloses a crystalline rasagiline tartrate salt, processes for the preparation, and pharmaceutical compositions thereof.

PCT Publication No. WO 2010/007181 (hereinafter referred to as the '181 application) discloses various solid state forms of rasagiline salts, including rasagiline benzoate crystalline form (Form I), rasagiline galactarate crystalline form (Form I), rasagiline gluconate amorphous form, rasagiline D-glucuronate amorphous form, rasagiline tosylate crystalline form (Form I), rasagiline phosphate amorphous form, rasagiline maleate crystalline form (Form I), rasagiline succinate crystalline form (Form I), rasagiline acetate crystalline forms (Forms I and II), rasagiline L-tartrate crystalline form (Form I), rasagiline hemitartrate crystalline form (Form I), rasagiline fumarate crystalline form (Form I), rasagiline hydrochloride crystalline forms (Forms I and II), and rasagiline besylate crystalline form (Form I); and characterizes them by powder X-ray diffraction (P-XRD) and IR spectroscopy; processes for their preparation; and pharmaceutical compositions thereof.

According to the '181 application, the rasagiline maleate crystalline Form I is characterized by an XRD pattern (2-theta) (±0.2 degrees) having characteristics peaks at 10.3, 12.0, 23.1, 24.1, 25.9 degrees with further peaks at 10.0, 12.4, 18.2, 18.7, 19.5, 20.7, 22.2, 23.6, 26.7 and 28.8 degrees.

As per the process exemplified in the '181 application, the rasagiline maleate crystalline Form I is prepared by dissolving rasagiline base in 2-propanol (6.4 volumes), followed by the addition of maleic acid and stirring the mixture for 2 hours at 40° C. The resulting mixture is allowed to cool to ambient temperature and stirred for 24 hours, and the mixture is filtered and dried under vacuum at 40° C.

The solvent medium, the volume of the solvent medium and the mode of isolation play very important roles in obtaining one solid state form over the other. There remains a need for novel solid state forms of rasagiline salts.

### SUMMARY

Solid state forms of the mandelate and salicylate salts of rasagiline have not been reported, isolated, or characterized in the literature.

The present inventors have now surprisingly and unexpectedly found solid state forms of rasagiline maleate, mandelate and salicylate salts with high purity, adequate stability, good flowability and good dissolution properties.

The novel solid state form of rasagiline maleate disclosed herein, designated herein as crystalline Form II, is characterized by an X-ray powder diffraction pattern having peaks expressed as 2-theta angle positions at about 7.66, 11.54, 13.19, 15.09, 15.76, 20.31, 21.0 and 21.52±0.2 degrees substantially as depicted in FIG. 1, which is different from the rasagiline maleate crystalline Form I reported in the '181 application. Further, rasagiline maleate Form II has adequate stability and good dissolution properties.

In one aspect, provided herein are novel solid state forms of a rasagiline salt, wherein the salt of rasagiline is a maleate salt, a mandelate salt or a salicylate salt.

In another aspect, rasagiline salts in a crystalline form are provided. In yet another aspect, the solid state forms of rasagiline salts exist in an anhydrous and/or solvent-free form or as a hydrate and/or a solvate form.

In another aspect, provided herein are novel solid state forms of a rasagiline salt, wherein the solid state form of rasagiline salt is a rasagiline maleate crystalline Form II, a rasagiline mandelate crystalline Form I, or a rasagiline salicylate crystalline Form I.

In another aspect, encompassed herein is a process for preparing the novel solid state forms of rasigiline salts comprising contacting rasagiline free base with an acid in a suitable solvent under suitable conditions to produce a reaction mass, and isolating the solid state form of rasagiline acid addition salt, wherein the solid state form of rasagiline salt is a rasagiline maleate crystalline Form II, a rasagiline mandelate crystalline Form I, or a rasagiline salicylate crystalline Form I.

In another aspect, provided herein is a pharmaceutical composition comprising a solid state form of rasagiline salt as disclosed herein, and one or more pharmaceutically acceptable excipients.

In still another aspect, provided herein is a pharmaceutical composition comprising a solid state form of a rasagiline salt made by the process disclosed herein, and one or more pharmaceutically acceptable excipients.

In still further aspect, encompassed herein is a process for preparing a pharmaceutical formulation comprising combining any one of the solid state forms of rasagiline salts disclosed herein with one or more pharmaceutically acceptable excipients.

In yet another aspect, provided herein is a method for treating a patient suffering from diseases caused by brain ischemia, a neurotoxic injury, head trauma injury, spinal trauma injury, symptoms of withdrawal from an addictive substance, or structural damage of the optic nerve; comprising administering a pharmaceutical composition comprising a novel solid state form of a rasagiline salt along with pharmaceutically acceptable excipients, wherein the solid state form of rasagiline salt is a rasagiline maleate crystalline Form II, a rasagiline mandelate crystalline Form I, or a rasagiline salicylate crystalline Form I.

In another aspect, the solid state forms of rasagiline salts disclosed herein for use in the pharmaceutical compositions have a D₉₀ particle size of less than or equal to about 500 microns, specifically about 1 micron to about 495 microns, and most specifically about 255 microns to about 490 microns.

### BRIEF DESCRIPTION OF THE DRAWINGS

FIG. 1 is a characteristic powder X-ray diffraction (XRD) pattern of Rasagiline maleate crystalline Form II.

FIG. 2 is a characteristic differential scanning calorimetric (DSC) thermogram of Rasagiline maleate crystalline Form II.

FIG. 3 is a characteristic powder X-ray diffraction (XRD) pattern of Rasagiline mandelate crystalline Form I.

FIG. 4 is a characteristic differential scanning calorimetric (DSC) thermogram of Rasagiline mandelate crystalline Form I.

FIG. 5 is a characteristic powder X-ray diffraction (XRD) pattern of Rasagiline salicylate crystalline Form I.

FIG. 6 is a characteristic differential scanning calorimetric (DSC) thermogram of Rasagiline salicylate crystalline Form I.

### DETAILED DESCRIPTION

In the formulation of drug compositions, it is important for the active pharmaceutical ingredient to be in a form in which it can be conveniently handled and processed. Convenient handling is important not only from the perspective of obtaining a commercially viable manufacturing process, but also from the perspective of subsequent manufacture of pharmaceutical formulations (e.g., oral dosage forms such as tablets) comprising the active pharmaceutical ingredient.

Chemical stability, solid state stability, and “shelf life” of the active pharmaceutical ingredient are important properties for a pharmaceutically active compound. The active pharmaceutical ingredient, and compositions containing it, should be capable of being effectively stored over appreciable periods of time, without exhibiting a significant change in the physico-chemical characteristics of the active pharmaceutical ingredient, e.g., its chemical composition, density, hygroscopicity and solubility. Thus, in the manufacture of commercially viable and pharmaceutically acceptable drug compositions, it is important, wherever possible, to provide the active pharmaceutical ingredient in a stable form.

New solid state forms of a pharmaceutical agent can further the development of formulations for the treatment of illnesses. For instance, solid forms of a compound are known in the pharmaceutical arts to affect, for example, the solubility, dissolution rate, bioavailability, chemical and physical stability, flowability, fractability, and compressibility of the compound, as well as the safety and efficacy of drug products based on the compound.

The discovery of novel salts in solid state form of pharmaceutically useful compounds provides a new opportunity to improve the performance characteristics of a pharmaceutical product. It also adds value to the material that a formulation scientist can use the same for designing, for example, a pharmaceutical dosage form of a drug with a targeted release profile or other desired characteristic.

According to one aspect, provided herein are novel solid state forms of a rasagiline salt, wherein the salt of rasagiline is a maleate salt, a mandelate salt or a salicylate salt.

In one embodiment, the solid state forms of rasagiline salts exist in a crystalline form. In yet another embodiment, the solid state forms of rasagiline salts exist in an anhydrous and/or solvent-free form, or as a hydrate and/or a solvate form. Such solvated or hydrated forms may be present as hemi-, mono-, sesqui-, di- or tri-solvates or hydrates. Solvates and hydrates may be formed as a result of the solvents used during the formation of the rasagiline salts becoming embedded in the solid lattice structure. Because formation of the solvates and hydrates occurs during the preparation of rasagiline salts, formation of a particular solvated or hydrated form depends greatly on the conditions and method used to prepare the salt. Solvents should be pharmaceutically acceptable.

According another one aspect, provided herein are novel solid state forms of a rasagiline salt, wherein the solid state form of a rasagiline salt is a rasagiline maleate crystalline Form II, a rasagiline mandelate crystalline Form I, or a rasagiline salicylate crystalline Form I.

In one embodiment, the solid state forms of rasagiline salts have the following characteristics, wherein:

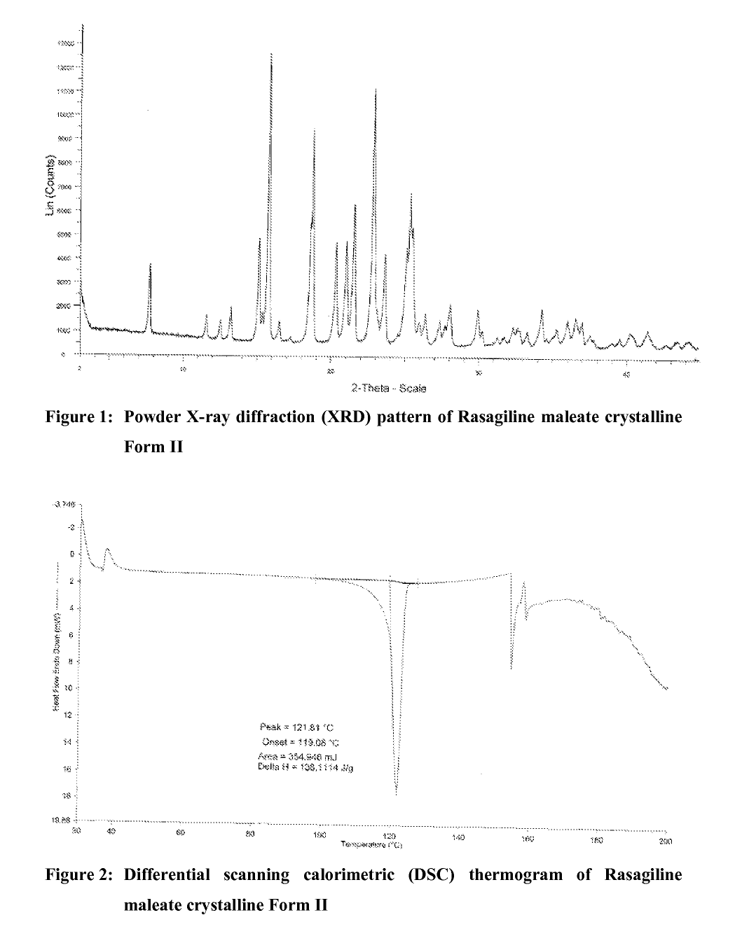

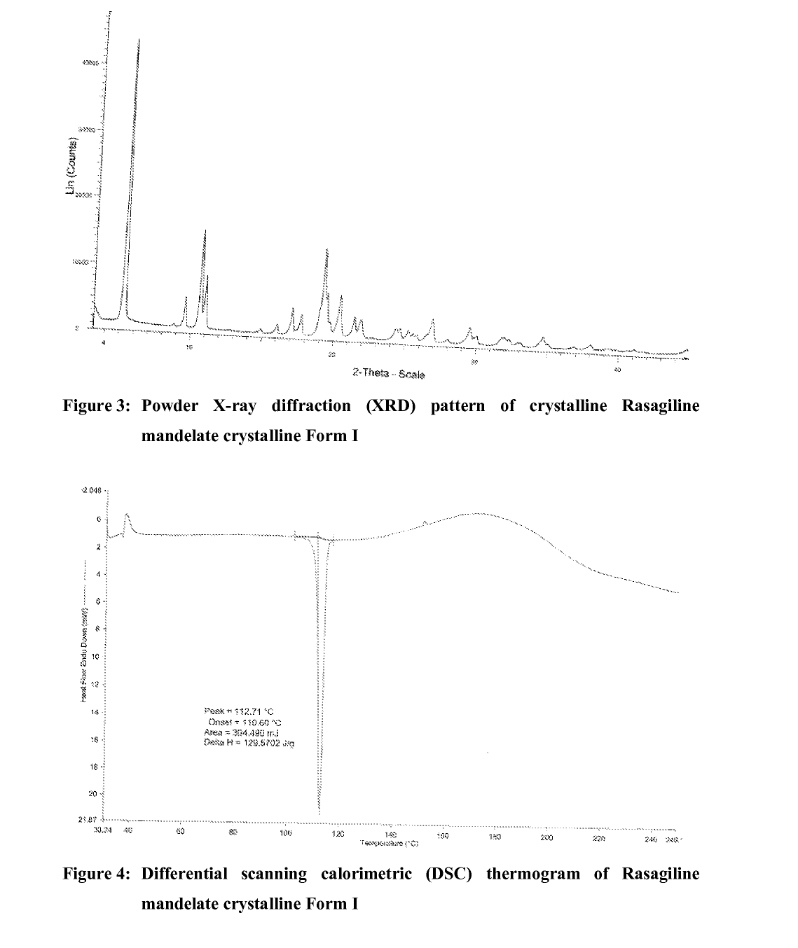

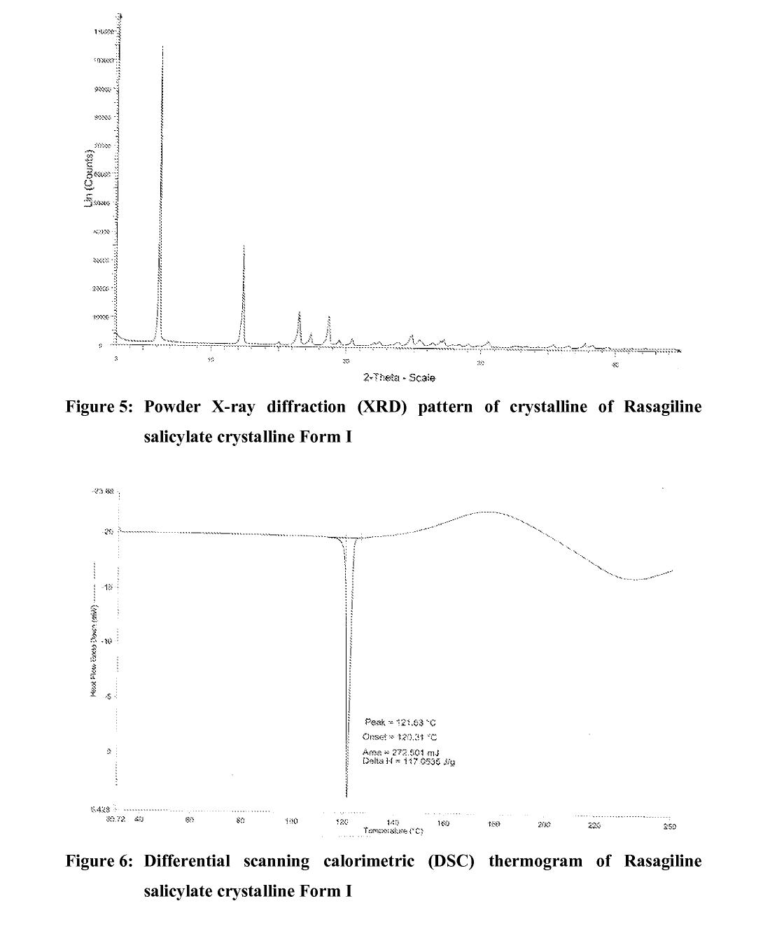

a) the rasagiline maleate crystalline Form II is characterized by one or more of the following properties:
i) a powder X-ray diffraction pattern substantially in accordance with FIG. 1;
ii) a powder X-ray diffraction pattern having peaks at about 7.66, 11.54, 13.19, 15.09, 15.76, 20.31, 21.0 and 21.52±0.2 degrees 2-theta;
iii) a powder X-ray diffraction pattern having further peaks at about 22.86, 25.11, 25.35, 26.38, 27.34, 28.06, 29.94, 30.23, 32.32 and 32.65±0.2 degrees 2-theta; and
iv) a differential scanning calorimetric (DSC) thermogram substantially in accordance with FIG. 2;
b) the rasagiline mandelate crystalline Form I is characterized by one or more of the following properties:
i) a powder X-ray diffraction pattern substantially in accordance with FIG. 3;
ii) a powder X-ray diffraction pattern having peaks at about 5.32, 10.67, 10.99, 19.31, 19.56 and 20.45±0.2 degrees 2-theta;
iii) a powder X-ray diffraction pattern having additional peaks at about 9.59, 17.12, 17.74, 21.48, 21.92, 24.41, 24.66, 26.95 and 29.57±0.2 degrees 2-theta; and
iv) a differential scanning calorimetric (DSC) thermogram substantially in accordance with FIG. 4; or
c) the rasagiline salicylate crystalline Form I is characterized by one or more of the following properties:
i) a powder X-ray diffraction pattern substantially in accordance with FIG. 5;
ii) a powder X-ray diffraction pattern having peaks at about 6.15, 12.35, 16.50, 17.36 and 18.70±0.2 degrees 2-theta;
iii) a powder X-ray diffraction pattern having additional peaks at about 19.46, 20.43, 22.44, 24.84, 25.46, 27.01, 27.23, 30.54 and 37.76±0.2 degrees 2-theta; and
iv) a differential scanning calorimetric (DSC) thermogram substantially in accordance with FIG. 6.

The solid state forms of rasagiline salts are stable, consistently reproducible, and are particularly suitable for bulk preparation and handling. Moreover, the solid state forms of rasagiline salts are useful intermediates in the preparation of rasagiline free base and its pharmaceutical acceptable salts thereof in high purity. The solid state forms of rasagiline salts have good flow properties and are far more stable at room temperature, enhanced temperature, at relative high humidities, and in aqueous media which makes them suitable for formulating.

According to another aspect, there is provided a process for the preparation of rasagiline maleate crystalline Form II, comprising:

a) heating a mixture containing maleic acid and isopropyl alcohol at reflux temperature to produce a hot solution;
b) adding a solution of rasagiline base in isopropyl alcohol to the hot solution obtained in step-(a) to produce a hot reaction mass containing rasagiline maleate, wherein the total amount of isopropyl alcohol employed for producing the hot reaction mass containing rasagiline maleate is in an amount of at least about 10 volumes per 1 gm of the rasagiline base;
c) cooling the reaction mass obtained in step-(b) gradually to a temperature of about 20° C. to 25° C. to produce a cooled reaction mass; and
d) recovering the pure crystalline Form II of rasagiline maleate from the cooled reaction mass obtained in step-(c).

In one embodiment, the total amount of isopropyl alcohol employed for producing the hot reaction mass containing rasagiline maleate obtained in step-(b) is about 15 volumes to about 25 volumes, and more specifically about 20 volumes, with respect to the rasagiline base.

According to another aspect, there is provided a process for the preparation of rasagiline mandelate crystalline Form I, comprising:

a) heating a mixture containing L-(+)-mandelic acid and isopropyl alcohol at reflux temperature to produce a hot solution;
b) adding a solution of rasagiline base in ethyl acetate to the hot solution obtained in step-(a) to produce a hot reaction mass;
c) cooling the hot reaction mass obtained in step-(b) gradually to a temperature of about 20° C. to 25° C. to produce a cooled reaction mass; and
d) recovering the pure crystalline Form I of rasagiline mandelate from the reaction mass obtained in step-(c).

According to another aspect, there is provided a process for the preparation of rasagiline salicylate crystalline Form I, comprising:

a) heating a mixture containing salicylic acid and a solvent at reflux temperature to produce a hot solution, wherein the solvent is selected from the group consisting of acetone, isopropyl alcohol, and mixtures thereof;
b) adding a solution of rasagiline base in ethyl acetate to the hot solution obtained in step-(a) to produce a hot reaction mass;
c) substantially removing the solvent from the hot reaction mass to obtain a residue;
d) combining the residue obtained in step-(c) with a solvent or a solvent mixture to produce a reaction mass, wherein the solvent is selected from the group consisting of isopropyl alcohol, diisopropyl ether, and mixtures thereof; and
e) recovering the pure crystalline Form I of rasagiline salicylate from the reaction mass obtained in step-(d).

The term “substantially removing” the solvent refers to at least 80%, specifically greater than about 85%, more specifically greater than about 90%, still more specifically greater than about 99%, and most specifically essentially complete (100%), removal of the solvent from the solvent solution.

Removal of solvent in step-(c) is accomplished, for example, by substantially complete evaporation of the solvent, concentrating the solution or distillation of solvent under inert atmosphere, or a combination thereof, to substantial elimination of total solvent present in the reaction mass.

The distillation process can be performed at atmospheric pressure or reduced pressure. Specifically, the distillation is carried out at a temperature of about 30° C. to about 110° C., more specifically at about 40° C. to about 90° C., and most specifically at about 45° C. to about 80° C.

Specifically, the solvent is removed at a pressure of about 760 mm Hg or less, more specifically at about 400 mm Hg or less, still more specifically at about 80 mm Hg or less, and most specifically from about 30 to about 80 mm Hg.

Combining of the residue with the solvent or the solvent mixture in step-(d) is done in a suitable order, for example, the residue is added to the solvent or the solvent mixture, or alternatively, the solvent or the solvent mixture is added to the residue. The addition is, for example, carried out drop wise or in one portion or in more than one portion. The addition is specifically carried out at a temperature of below about 100° C., more specifically at about 20° C. to about 80° C. under stirring. After completion of addition process, the resulting mass is optionally stirred at a temperature of about 25° C. to about 100° C. for at least 1 hour and specifically at a temperature of about 25° C. to about 50° C. for about 10 to about 4 days to produce the reaction mass.

As used herein, “reflux temperature” means the temperature at which the solvent or solvent system refluxes or boils at atmospheric pressure.

The recovering of solid state forms of rasagiline salt is carried out by methods such as filtration, filtration under vacuum, decantation, centrifugation, or a combination thereof. In one embodiment, solid state form of rasagiline salt is recovered by filtration employing a filtration media of, for example, a silica gel or celite.

The processes can produce the solid state forms of rasagiline salts in substantially pure form.

The term “substantially pure solid state form of rasagiline salt” refers to the solid state form of rasagiline salt having a purity of greater than about 98 wt %, specifically greater than about 99 wt %, more specifically greater than about 99.5 wt %, and still more specifically greater than about 99.9 wt %. The purity is preferably measured by High Performance Liquid Chromatography (HPLC). For example, the purity of the solid state form of rasagiline salt obtained by the process disclosed herein can be about 98% to about 99.95%, or about 99% to about 99.99%, as measured by HPLC.

In one embodiment, the process disclosed herein provides stable solid state forms of rasagiline salts. The term “stable solid state form” refers to stability of the solid state form under the standard temperature and humidity conditions of testing of pharmaceutical products, wherein the stability is indicated by preservation of the original solid state form.

The substantially pure solid state form of rasagiline salt obtained by above processes may be further dried in, for example, a Vacuum tray dryer, a Rotocon vacuum dryer, a Vacuum paddle dryer or a pilot plant Rota vapor, to further lower residual solvents. Drying can be carried out under reduced pressure until the residual solvent content reduces to the desired amount such as an amount within the limits given by the International Conference on Harmonization of Technical Requirements for Registration of Pharmaceuticals for human use (ICH) guide lines.

In one embodiment the drying is carried out at atmospheric pressure or reduced pressures, such as below about 200 mm Hg, or below about 50 mm Hg, at temperatures such as 35° C. to about 80° C. The drying can be carried out for any desired time period that achieves the desired result, such as about 1 to 20 hours. Drying may also be carried out for shorter or longer periods of time depending on the product specifications. Temperature and pressure are chosen based on the volatility of the solvent being used and the foregoing should be considered as only a general guidance. Dying can be suitably carried out in a tray dryer, a vacuum oven, an air oven, or using a fluidized bed drier, a spin flash dyer, a flash dryer and the like. Drying equipment selection is well within the ordinary skill in the art.

The solid state form of rasagiline salt obtained by the processes disclosed herein is further optionally converted into rasagiline free base or its pharmaceutically acceptable salts by treating the solid state form of rasagiline salt with a base and/or a respective acid.

Further encompassed herein is the use of the solid state form of a rasagiline salt for the manufacture of a pharmaceutical composition together with a pharmaceutically acceptable carrier, wherein the solid state form of rasagiline salt is a rasagiline maleate crystalline Form II, a rasagiline mandelate crystalline Form I or a rasagiline salicylate crystalline Form I.

A specific pharmaceutical composition of the solid state form of rasagiline salt is selected from a solid dosage form and an oral suspension.

In one embodiment, the solid state form of rasagiline salt has a D₉₀ particle size of less than or equal to about 500 microns, specifically about 1 micron to about 495 microns, and most specifically about 255 microns to about 490 microns, wherein the solid state form of rasagiline salt is a rasagiline maleate crystalline Form II, a rasagiline mandelate crystalline Form I, or a rasagiline salicylate crystalline Form I.

In another embodiment, the particle sizes of the solid state form of rasagiline salt are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided pharmaceutical compositions comprising the solid state form of rasagiline salt and one or more pharmaceutically acceptable excipients, wherein the solid state form of rasagiline salt is a rasagiline maleate crystalline Form II, a rasagiline mandelate crystalline Form I, or a rasagiline salicylate crystalline Form I.

According to another aspect, there is provided pharmaceutical compositions comprising the solid state form of rasagiline salt prepared according to process disclosed herein and one or more pharmaceutically acceptable excipients, wherein the solid state form of rasagiline salt is a rasagiline maleate crystalline Form II, a rasagiline mandelate crystalline Form I, or a rasagiline salicylate crystalline Form I.

According to another aspect, there is provided a process for preparing a pharmaceutical formulation comprising combining the solid state form of rasagiline salt prepared according to processes disclosed herein, with one or more pharmaceutically acceptable excipients, wherein the solid state form of rasagiline salt is a rasagiline maleate crystalline Form II, a rasagiline mandelate crystalline Form I, or a rasagiline salicylate crystalline Form I.

According to another aspect, there is provided a method for treating a patient suffering from diseases caused by brain ischemia, a neurotoxic injury, head trauma injury, spinal trauma injury, symptoms of withdrawal from an addictive substance, or structural damage of the optic nerve; comprising administering a pharmaceutical composition comprising the solid state form of a rasagiline salt along with pharmaceutically acceptable excipients, wherein the solid state form of rasagiline salt is a rasagiline maleate crystalline Form II, a rasagiline mandelate crystalline Form I, or a rasagiline salicylate crystalline Form I.

Yet in another embodiment, pharmaceutical compositions comprise at least a therapeutically effective amount of solid state form of a rasagiline salt, wherein the solid state form of rasagiline salt is a rasagiline maleate crystalline Form II, a rasagiline mandelate crystalline Form I or a rasagiline salicylate crystalline Form I. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The solid state form of rasagiline salt may also be administered as suppositories, ophthalmic ointments and suspensions, and parenteral suspensions, which are administered by other routes, wherein the solid state form of rasagiline salt is a rasagiline maleate crystalline Form II, a rasagiline mandelate crystalline Form I, or a rasagiline salicylate crystalline Form I.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described herein.

In one embodiment, capsule dosage forms contain solid state form of rasagiline salt within a capsule which may be coated with gelatin. Tablets and powders may also be coated with an enteric coating. Suitable enteric coating include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such as calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### Instrumental Details:

### X-Ray Powder Diffraction (P-XRD):

The X-Ray powder diffraction was measured by an X-ray powder diffractometer equipped with a Cu-anode (λ=1.54 Angstrom), X-ray source was operated at 40 kV, 40 mA and a Ni filter was used to strip K-beta radiation. Two-theta calibration was performed using an NIST SRM 1976, Corundum standard. The sample was analyzed using the following instrument parameters: measuring range=3-45° 2-theta; step width=0.01579°; and measuring time per step=0.11 second.

### Differential Scanning Calorimetry (DSC):

Differential Scanning calorimetry (DSC) measurements were performed with a Differential Scanning calorimeter (DSC Q 1000 V23.5 Build 72, Universal V4.3A TA Instruments) at a scan rate of 10° C. per minute.

The following examples are given for the purpose of illustrating the present disclosure and should not be considered as limitation on the scope or spirit of the disclosure.

### EXAMPLES

### Example 1

### Process for Preparing Rasagiline Maleate Crystalline Form II

A mixture of isopropyl alcohol (30 ml) and maleic acid (2.23 g) was heated to reflux temperature of about 80 to 83° C. A solution of rasagiline base (3 g) dissolved in isopropyl alcohol (30 ml) was added to the hot solution. The reaction mass was cooled gradually to 20 to 25° C., and the solid was filtered. The solid obtained was further washed with chilled isopropyl alcohol (10 ml) and dried in vacuum oven at 50 to 55° C. for 8 hours to yield 2.1 g of rasagiline maleate crystalline Form II.

### Example 2

### Process for Preparing Rasagiline Mandelate Crystalline Form I

A mixture of isopropyl alcohol (30 ml) and L-(+)-mandelic acid (2.9 g) was heated to reflux temperature of about 80 to 83° C. A solution of rasagiline base (3 g) dissolved in ethyl acetate (30 ml) was added to the hot solution. The reaction mass was cooled gradually to about 20 to 25° C. and stirred for 24 hours at 25 to 30° C. The resulting solid was filtered, washed with chilled isopropyl alcohol (10 ml) and then dried in vacuum oven at 50 to 55° C. for 8 hours to yield 3.9 g of rasagiline mandelate crystalline Form I.

### Example 3

### Process for Preparing Rasagiline Salicylate Crystalline Form I

A mixture of acetone (20 ml) and salicylic acid (2.6 g) was heated to reflux temperature, followed by the addition of a solution of rasagiline base (3 g) dissolved in ethyl acetate (30 ml). The solvent was distilled completely under vacuum. Isopropyl alcohol (10) and diisopropyl ether (20 ml) were added to the resulting residue and stirred for 4 days. The separated solid was filtered, washed with chilled diisopropyl ether (5 ml) and then dried in vacuum oven at 50 to 55° C. for 8 hours to yield 3.5 g of rasagiline salicylate crystalline Form I.

Unless otherwise indicated, the following definitions are set forth to illustrate and define the meaning and scope of the various terms used to describe the invention herein.

The term “crystalline form” refers to a crystal modification that can be characterized by analytical methods such as X-ray powder diffraction, IR-spectroscopy, differential scanning calorimetry (DSC) or by its melting point.

The term “pharmaceutically acceptable” means that which is useful in preparing a pharmaceutical composition that is generally non-toxic and is not biologically undesirable, and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dihydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Solid state form of a rasagiline salt, wherein the salt of rasagiline is a mandelate salt, a salicylate salt, or a rasagiline maleate salt crystalline Form II.

2. The solid state form of rasagiline salt of claim 1, having the following characteristics, wherein:
a) the rasagiline maleate crystalline Form II is characterized by one or more of the following properties:
i) a powder X-ray diffraction pattern substantially in accordance with FIG. 1;
ii) a powder X-ray diffraction pattern having peaks at about 7.66, 11.54, 13.19, 15.09, 15.76, 20.31, 21.0 and 21.52±0.2 degrees 2-theta;
iii) a powder X-ray diffraction pattern having further peaks at about 22.86, 25.11, 25.35, 26.38, 27.34, 28.06, 29.94, 30.23, 32.32 and 32.65±0.2 degrees 2-theta; and
iv) a differential scanning calorimetric (DSC) thermogram substantially in accordance with FIG. 2;
b) the solid state form of rasagiline mandelate, which is in a crystalline Form I, is characterized by one or more of the following properties:
i) a powder X-ray diffraction pattern substantially in accordance with FIG. 3;
ii) a powder X-ray diffraction pattern having peaks at about 5.32, 10.67, 10.99, 19.31, 19.56 and 20.45±0.2 degrees 2-theta;
iii) a powder X-ray diffraction pattern having additional peaks at about 9.59, 17.12, 17.74, 21.48, 21.92, 24.41, 24.66, 26.95 and 29.57±0.2 degrees 2-theta; and
iv) a differential scanning calorimetric (DSC) thermogram substantially in accordance with FIG. 4; and
c) the solid state form of rasagiline salicylate, which is in a crystalline Form I, is characterized by one or more of the following properties:
i) a powder X-ray diffraction pattern substantially in accordance with FIG. 5;
ii) a powder X-ray diffraction pattern having peaks at about 6.15, 12.35, 16.50, 17.36 and 18.70±0.2 degrees 2-theta;
iii) a powder X-ray diffraction pattern having additional peaks at about 19.46, 20.43, 22.44, 24.84, 25.46, 27.01, 27.23, 30.54 and 37.76±0.2 degrees 2-theta; and
iv) a differential scanning calorimetric (DSC) thermogram substantially in accordance with FIG. 6.

3. A process for the preparation of rasagiline maleate crystalline Form II of claim 1, comprising:
a) heating a mixture containing maleic acid and isopropyl alcohol at reflux temperature to produce a hot solution;
b) adding a solution of rasagiline base in isopropyl alcohol to the hot solution obtained in step-(a) to produce a hot reaction mass containing rasagiline maleate, wherein the total amount of isopropyl alcohol employed for producing the hot reaction mass containing rasagiline maleate is in an amount of at least about 10 volumes per 1 gm of the rasagiline base;
c) cooling the hot reaction mass obtained in step-(b) gradually to a temperature of about 20° C. to 25° C. to produce a cooled reaction mass; and
d) recovering the pure crystalline Form II of rasagiline maleate from the cooled reaction mass obtained in step-(c).

4. The process of claim 3, wherein the total amount of isopropyl alcohol employed for producing the hot reaction mass containing rasagiline maleate obtained in step-(b) is about 15 volumes to about 25 volumes with respect to the rasagiline base.

5. The process of claim 4, wherein the total amount of isopropyl alcohol employed is about 20 volumes with respect to the rasagiline base.

6. A process for the preparation of solid state form of rasagiline mandelate of claim 1, comprising:
a) heating a mixture containing L-(+)-mandelic acid and isopropyl alcohol at reflux temperature to produce a hot solution;
b) adding a solution of rasagiline base in ethyl acetate to the hot solution obtained in step-(a) to produce a hot reaction mass;
c) cooling the hot reaction mass obtained in step-(b) gradually to a temperature of about 20° C. to 25° C. to produce a cooled reaction mass; and
d) recovering the pure crystalline Form I of rasagiline mandelate from the cooled reaction mass obtained in step-(c).

7. A process for the preparation of solid state form of rasagiline salicylate of claim 1, comprising:
a) heating a mixture containing salicylic acid and a solvent at reflux temperature to produce a hot solution, wherein the solvent is selected from the group consisting of acetone, isopropyl alcohol, and mixtures thereof;
b) adding a solution of rasagiline base in ethyl acetate to the hot solution obtained in step-(a) to produce a hot reaction mass;
c) substantially removing the solvent from the hot reaction mass to obtain a residue;
d) combining the residue obtained in step-(c) with a solvent or a solvent mixture to produce a reaction mass, wherein the solvent is selected from the group consisting of isopropyl alcohol, diisopropyl ether, and mixtures thereof; and
e) recovering the pure crystalline Form I of rasagiline salicylate from the reaction mass obtained in step-(d).

8. The process of claim 7, wherein the removal of solvent in step-(c) is accomplished by substantially complete evaporation of the solvent, concentrating the solution or distillation of solvent under inert atmosphere, or a combination thereof.

9. The process of any one of claim 3, wherein the recovering is carried out by filtration, filtration under vacuum, decantation, centrifugation, filtration employing a filtration media of a silica gel or celite, or a combination thereof; and wherein the crystalline form of rasagiline salt obtained is further dried under vacuum or at atmospheric pressure, at a temperature of about 35° C. to about 80° C.

10. A pharmaceutical composition comprising solid state form of a rasagiline salt and one or more pharmaceutically acceptable excipients, wherein the solid state form of rasagiline salt is a rasagiline maleate crystalline Form II, a rasagiline mandelate crystalline Form I, or a rasagiline salicylate crystalline Form I.

11. The pharmaceutical composition of claim 10, wherein the pharmaceutical composition is a solid dosage form, an oral suspension, a liquid, a powder, an elixir, an aerosol, a syrup, or an injectable solution.

12. The pharmaceutical composition of claim 10, wherein the solid state form of rasagiline salt has a D₉₀ particle size of less than or equal to about 500 microns.

13. The pharmaceutical composition of claim 12, wherein the D₉₀ particle size is about 1 micron to about 495 microns.

14. The pharmaceutical composition of claim 13, wherein the D₉₀ particle size is about 255 microns to about 490 microns.

15. A method for treating a patient suffering from diseases caused by brain ischemia, a neurotoxic injury, head trauma injury, spinal trauma injury, symptoms of withdrawal from an addictive substance, or structural damage of the optic nerve; comprising administering a pharmaceutical composition comprising the solid state form of a rasagiline salt along with pharmaceutically acceptable excipients, wherein the solid state form of rasagiline salt is a rasagiline maleate crystalline Form II, a rasagiline mandelate crystalline Form I, or a rasagiline salicylate crystalline Form I.

16. The process of claim 6, wherein the recovering is carried out by filtration, filtration under vacuum, decantation, centrifugation, filtration employing a filtration media of a silica gel or celite, or a combination thereof; and wherein the crystalline form of rasagiline salt obtained is further dried under vacuum or at atmospheric pressure, at a temperature of about 35° C. to about 80° C.

17. The process of claim 7, wherein the recovering is carried out by filtration, filtration under vacuum, decantation, centrifugation, filtration employing a filtration media of a silica gel or celite, or a combination thereof; and wherein the crystalline form of rasagiline salt obtained is further dried under vacuum or at atmospheric pressure, at a temperature of about 35° C. to about 80° C.
